(19) United States
(12) Patent Application Publication
(10) Pub. No.: US 2022/0175305 A1
(43) Pub. Date: Jun. 9, 2022

(54) METHOD FOR DIAGNOSING NEUROLOGICAL DISEASES THROUGH MEASURING TYPING ERRORS

(71) Applicant: NeuraMetrix, Inc., San Francisco, CA (US)

(72) Inventor: Jan SAMZELIUS, San Francisco, CA (US)

(73) Assignee: NeuraMetrix, Inc., San Francisco, CA (US)

(21) Appl. No.: 17/497,803

(22) Filed: Oct. 8, 2021

Related U.S. Application Data
(60) Provisional application No. 63/089,514, filed on Oct. 8, 2020.
(60) Provisional application No. 63/089,511, filed on Oct. 8, 2020.

(51) Int. Cl. A61B 5/00; A61B 5/16

(52) U.S. Cl. CPC A61B 5/4082; A61B 5/16; A61B 5/7475

(57) ABSTRACT

A typing data analysis system may be used to accurately detect early onset of CNS diseases and psychiatric disorders based on typing data. The typing data analysis system may determine typing cadence data and typing error data from typing data captured during normal, everyday typing activities including typing an email, blog, or text message. The typing cadence data and typing error data may be analyzed to determine typing based measures of different CNS diseases and psychiatric disorders. Typing based measures may be continuously determined from a patient's typing activities on the patient's personal computer, smartphone, or other commonly used device to monitor the patient's brain function over time. The typing based measures for the patient may be compared to profiles of CNS diseases and/or psychiatric disorders to screen the patient for a particular disease/disorder.

### PRIORITY CLAIM

This application claims priority under 35 USC 120 and claims the benefit under 35 USC 119(e) to U.S. Provisional Patent Application Ser. No. 63/089,514, filed Oct. 8, 2020 and entitled “Method For Diagnosing Neurological Diseases Through Measuring Typing Errors” and to U.S. Provisional Patent Application Ser. No. 63/089,511, filed Oct. 8, 2020 and entitled “Method For High Accuracy Diagnose Of Brain Diseases and Psychiatric Disorders”, the entirely of which are incorporated herein by reference.

### APPENDICES

Appendix A (14 pages) contains an example of a portion of the key action data stream for a particular user (serial number 1234); and

Appendix B (1 page) contains an example of a piece of text that was typed by a user that generated the portion of the key action data stream shown in Appendix A.

Appendix A and Appendix B are part of the specification and are incorporated herein by reference.

### FIELD

The disclosure relates generally to a system and method for diagnosing central nervous system diseases and psychiatric disorders based on typing errors.

### BACKGROUND

Neurological diseases begin to affect the human body years or even decades before the onset of symptoms observable by the patient or in the clinic. Patients of brain diseases often report that they noticed symptoms and changes years before diagnosis. For example, reduced quality of handwriting often precedes a diagnosis of Alzheimer's. Parkinson's patients mention that more and different typing errors occurred well before the clinical diagnosis.

Neurological diseases, such as Alzheimer's and Parkinson's, are very difficult to correctly diagnose, since many symptoms are the same for several diseases and many measurements used in the clinics for different diseases are quite similar. The weaknesses of measurements currently used in clinics make early detection nearly impossible and optimizing the disease management (drug regimen, diet, exercise, etc.) very difficult. Existing measures are simply not granular enough to pick up very early signs of disease or measure the right thing in order to perform early detection. Similarly, the tools currently used by physicians cannot provide rapid feedback on changes in disease management. Patients are typically only examined and/or tested 1-4 times per year therefore it is difficult to establish a baseline neurological function and accurately monitor changes with so few data points. Additionally, many of the measurements provided by current testing methods cannot be repeated frequently, due to learning effects.

Furthermore, the tools used by neurologists and other healthcare providers today are not sensitive enough to ensure that the patient is given an accurate diagnosis. Estimates have shown that the rate of misdiagnosis of Central Nervous System (“CNS”) diseases (such as Alzheimer's, Parkinson's, MS and Huntington's, and also including psychiatric disorders, such as depression) is 40%. In many cases, the misdiagnosis means that the patient is given incorrect medication or that treatment of the patient's actual disease is delayed until symptoms are more clearly visible. It is desirable to provide a system that provides continuous monitoring of CNS diseases.

One known technique to monitor CNS diseases is by measurement and analyzing typing cadence which is the rhythm with which each user type of a keyboard. The typing cadence is unique to each user and also fairly consistent under normal conditions. However, when a user has a CNS disease, the typing cadence of the user can change and those inconsistencies in typing cadence can be used to detect the CNS diseases at an early stage. While detecting this inconsistency is valuable, it is desirable to provide a system and method that provides a more immediate tangible measurement that can be used to detect and monitor CNS diseases.

### BRIEF DESCRIPTION OF THE DRAWINGS

FIG. 1 illustrates an example of an implementation of a system for diagnosing and monitoring a neurological disorder;

FIG. 2 illustrates an example of an implementation of a computing device that is part of the system for diagnosing and monitoring a neurological disorder;

FIG. 3 illustrates an example of an implementation of a backend component that is part of the system for diagnosing and monitoring a neurological disorder;

FIG. 4 illustrates an example of the data generated by the system shown in FIG. 1;

FIG. 5 illustrates an example of the processes performed by each computing device of the system for diagnosing and monitoring a neurological disorder;

FIG. 6 illustrates an example of the processes performed by the backend component of the system for diagnosing and monitoring a neurological disorder;

FIG. 7 illustrates a system for diagnosing and monitoring a neurological disorder with a first embodiment of a continuous monitoring component;

FIG. 8 illustrates a second embodiment of the continuous monitoring system that is a standalone system;

FIG. 9 illustrates further details of the continuous monitoring component shown in FIGS. 7 and 8;

FIG. 10 illustrates a continuous monitoring method that may use the continuous monitoring component; and

FIG. 11 shows an example of a chart generated by the system.

### DETAILED DESCRIPTION OF ONE OR MORE EMBODIMENTS

The disclosure is particularly applicable to the detection or continuous monitoring of CNS diseases using the implementation of the typing data analysis system described below that measures typing errors and it is in this context that the disclosure will be described. It will be appreciated, however, that the system and method has greater utility since the diagnoses of CNS diseases may be implemented using other implementations of the typing cadence system and typing error analysis that are within the scope of the disclosure. Furthermore, the typing error system and method may be used to detect/monitor other conditions (medical or otherwise) of the user that cause different types of typing errors or a larger number of typing errors. In all cases, the detected typing errors provide a more immediate, tangible measurement as compared to the typing cadence inconsistency described above.

The system and method for the diagnosis of CNS diseases uses typing errors (e.g., how often a person makes typing errors, distinguishing by the type of error made) generally and at a particular cadence and typing cadence, the rhythm with which we all type on a keyboard, and to provide continuous monitoring of neural function so that the performance of the brain in terms of consistency can be measured objectively, precisely and at incredibly granularity. At one level of analysis, the system and method uses typing cadence (TC) which is a very strong habit. Research has shown that it is one of our strongest habits and habits are hardwired in the brain and when the brain is attacked by disease, the hardwiring begins to break, but it breaks slowly and in small increments which are not visible to a clinic, but visible using the continuous monitoring of CNS diseases using typing cadence. The typing data analysis system augments the typing cadence data analysis with a second level of analysis based on typing errors. Identifying and monitoring typing errors that occur during normal typing activities (e.g., continuously typing an email or a text message) adds another layer of analysis that improves the accuracy of CNS disease diagnosis.

The two tiered approach to diagnosis of CNS diseases and psychiatric disorders is data intensive and involves continuous monitoring of normal, everyday typing activities instead of targeted testing activities in clinical settings. TC produces lots of data, can easily be based on natural behavior, not testing behavior and is extremely user friendly to administer (i.e., TC data may be collected during normal every day typing activities that occur at home on a personal computer). Typing error data augments the TC dataset with additional features of a person's typing behavior. These additional features provide additional data that can be analyzed to help tease out subtle changes in typing behavior at a very granular level that are required for early detection of CNS diseases. Features extracted from typing error data may also be added to TC based CNS disease profiles to generate more detailed profiles of each CNS disease that may be used to more accurate diagnoses of CNS diseases.

To diagnose CNS disease based on typing data, TC and typing error data may be collected for a group of people known to have a particular CNS disease. The TC and typing error data may be collected overtime to capture data throughout the initial onset and development of the disease. The combination of the collected TC and typing error data may be used to determine a detailed signature or fingerprint for each disease. For example, the TC and typing error data measurement reliably generates sufficient sample on over 400 binary variables for TC and hundreds of different typing error sequences and multiple error based variables for typing error data. Each CNS disease and psychiatric disorder and version of disease or disorder thereof uniquely manifests itself in the 400 variables, the typing error sequences, and typing error variables. For example, Parkinson's patients type well in the middle of the keyboard, but have difficulty typing the keys at the perimeter of the keyboard. Accordingly, the typing error variables for Parkinson's disease may include more typing errors including sequences of presses of keys at the edges of the keyboard. In many keyboards, the backspace key is located toward the edge of the keyboard, therefore typing error variables for Parkinson's disease may also include longer dwell times and or longer flight times for presses of the backspace key and or more inconsistency in overall typing behavior relative to other CNS diseases. The variables associated with each CNS disease and/or psychiatric disorder may be measures of a particular disease/disorder. The measures determined from the TC data and the typing error data be complied in profile that is specific to each disease/disorder. The TC and typing error data for a patient may then be compared to the TC and typing error data included in the profiles of various CHS diseases to screen the patient for each disease.

For example, the TC and typing error based measures for a particular CNS disease may be determined from the typing data of a group of individuals with the particular CNS disease. The measures for the CNS disease may be compared to the same set of measures determined from the TC and typing error data of a group of individuals without such disease. The comparison may be done on a measure by measure basis, to determine which measures are most significant. The most significant measures for the CNS disease are then stored in a profile for the CNS disease and form the basis of comparison for the purposes of diagnosing the particular CNS disease. To screen a patient for the early onset of a CNS disease, the number of errors, type of errors, typing cadence, and other measures of the CNS disease determined from the typing data of the patient are compared to the values of each of these measures in the profile for the CNS disease. If the measures determined from the patient's typing data meet or exceed the measures in the CNS disease profile, the patient may be diagnosed with the CNS disease and/or recommended for more extensive testing, such as MM and other brain imaging techniques.

In various embodiments, a patient's typing error measures may be compared to the typing error measures of groups having a similar cadence to the patient. For example, the measures determined from typing error data (e.g., a number errors, frequency of errors, and/or a type for each error) for an individual having a particular ‘s typing cadence may be compared to typing error measures determined from typing data for groups of individuals that have a similar typing cadence and have a CNS disease and individuals that have a similar typing cadence and do not have a CNS disease. Patients having typing error measures that are similar to (e.g., are within a defined similarity threshold) the group of individuals with a similar typing cadence that have a CNS disease may be diagnosed with the CNS disease and/or recommended for more extensive testing, such as Mill and other brain imaging techniques. Patients that have typing error measures that are similar to (e.g., are within a defined similarity threshold) groups of individuals with a similar typing cadence that do not have a CNS disease may identified as without such disease.

In various embodiments, an individual's typing error and TC measures may be compared overtime. For example, the measures determined from the individual's TC data and typing error data may be compared to measures determined from the individual's TC and typing error data for previous time periods to reveal key changes in speed and typing consistency. The changes in speed and typing consistency observed the in the individual may be compared to like measures stored in profiles of CNS diseases and psychiatric disorders to screen the individual for a particular disease/disorder.

The techniques for monitoring patient typing data and diagnosing CNS diseases based on TC and typing error data deliver a dramatic improvement in the accuracy of diagnoses. The resulting increase in diagnostic accuracy improves patient quality of life by accelerating disease treatment and providing lower costs to heath care systems by reducing the amount of testing required to make an accurate disease diagnosis. The techniques for monitoring patient TC and typing error data also allow much better tests of the efficacy of drug regimens or other possible ways to reduce the speed of development of these neurological diseases. By tracking a patient's brain function over time, the monitoring techniques provide suppliers of such medical solutions a very simple and easy way to test the effect of a solution. Now, an implementation of a system for detecting and monitoring a neurological disorder that may be part of the system and method for high accuracy diagnosis of brain diseases and psychiatric disorders is described.

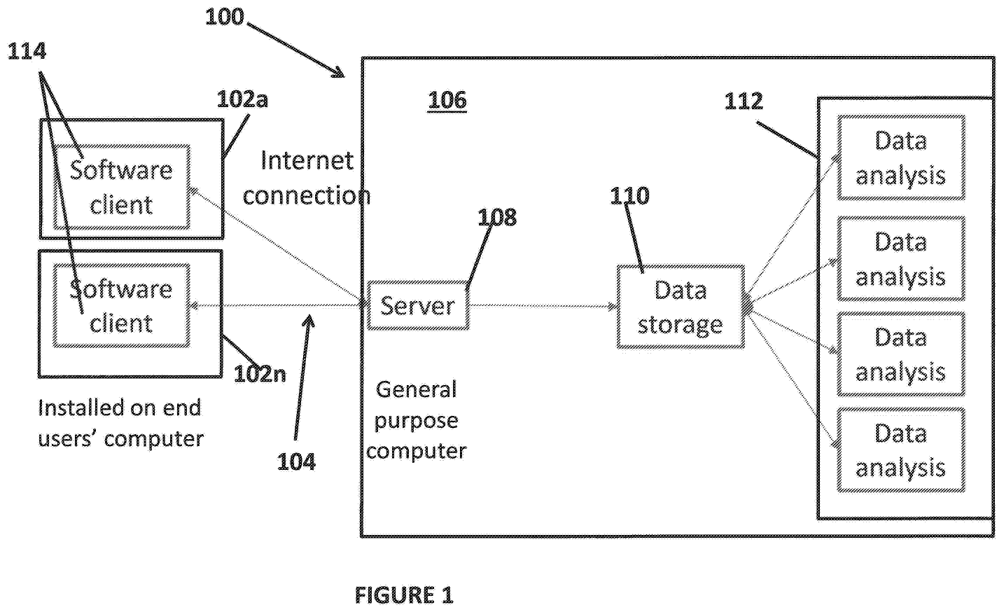

FIG. 1 illustrates an example of an implementation of a system 100 for detecting and monitoring a neurological disorder. The system shown in FIG. 1 is implemented using a client server architecture, but the system may also be implemented as a cloud computing architecture, a standalone computer architecture or a software as a service (SaaS) model in which the typing cadence and the typing error component, for example, may be downloaded to each computing device as needed. The system 100 may have one or more computing devices 102, such as computing device 102a, . . . , computing device 102n, each of which are connect to and communicate over a communications path 104 with a backend component 106. Each computing device 102 gathers, stores and communicates typing cadence data and/or typing error data about a user who uses the computing device and the typing cadence data and/or typing error data is sent over the communications path 104 to the backend component 106 that receives, stores and analyzes the typing cadence data and/or the typing error data to detect symptoms of a neurological disorder.

Each computing device may be a processor based system that has some input device so that the computing device is capable of collecting typing cadence data and typing error data from the user as the user performs his daily tasks that include typing on the input device. For example, each computing device may be a desktop computer, a laptop computer, a tablet computer, a smartphone, such as an Apple iPhone product or an Android Operating system (OS) based device, or a traditional mobile phone. The input devices for each computing device may include, for example, a built-in keyboard, a detachable keyboard, a glass surface, a touchscreen, a virtual keyboard, a keypad, an electronically generated keyboard on a touchscreen and the like. Alternatively, each computing device may be a standalone device that captures typing cadence and/or typing errors of a user. Each computing device may also have a typing cadence and typing error component 114 that may be resident on each computing device.

The typing cadence and typing error component 114 may be a hardware circuit, a piece of software code, a hardware circuit programmed with a plurality of lines of computer code or an application. In the case of the typing cadence and typing error component 114 having software code or computer code, the typing cadence and typing error component 114 may be stored in a memory of the computing device and may be executed by a processor of the computing device 102. The typing cadence and typing error component 114 may perform a series of processes of the algorithm that provides a recording of typing data (e.g., key action data) and determines typing cadence data and typing error data from the recorded typing data.

The communications path 104 may be, as shown in FIG. 1, a public internet connection or a private network connection. Each computing device 102 may connect to the communications path using a known protocol and then connect to another system, such as the backend component and communicate with the backend component using a known protocol that may or may not be secure. For example, the communications path 104 may be a computer network, the Ethernet, the Internet, a digital data network, a wireless digital data network and the like. In one implementation, the communication path between each computing device and the backend component may be a standard internet connection (secure or public) while the communication path between the front-end components 108 and the storage units 110 may be implemented using a dedicated, private connection.

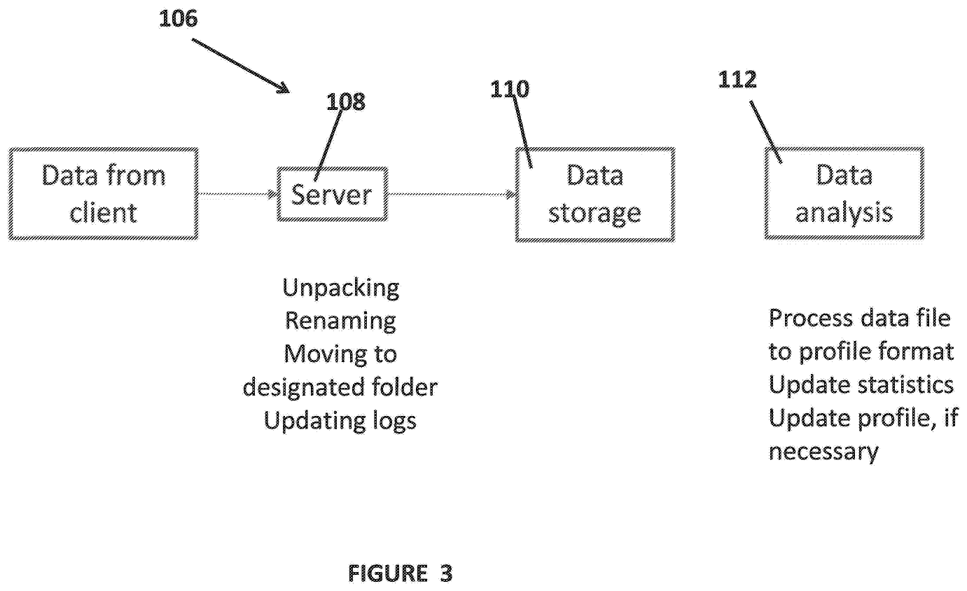

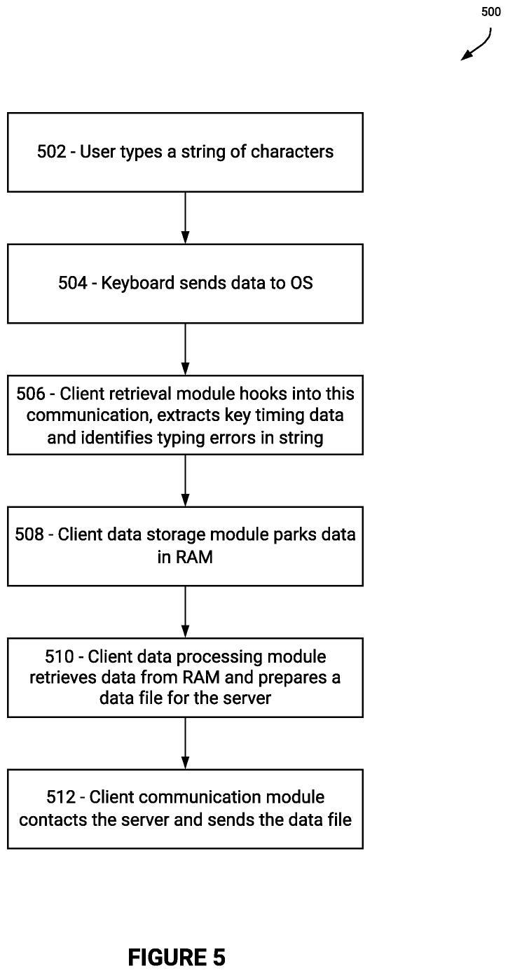

The backend component 106 may include one or more front end components 108, one or more data stores 110 and one or more data analytics components 112 that are connected to each other as shown in FIG. 1. These components of the backend component 106 may be implemented in hardware or software or a combination of hardware and software and may perform a series of processes of the algorithm described below in FIG. 5. In one implementation, the components are implemented in computing resources, such as one or more server computers or one or more cloud computing resources that have at least a processor and a memory. In that implementation, each component may include a plurality of lines of computer code that may be stored in the memory and executed by the processor to provide the functions of each component that are described in more detail below with reference to FIG. 3.

In operation in one implementation, every user, patient or control, has the typing cadence and typing error component 114 installed on their computing device 102. To generate typing cadence data, the typing cadence and typing error component 114 hooks into the operating system and taps into the data stream from the input device and copies the clock time data for each key action/event. Each key action/event may be a pressing of a key (key press) or a releasing of the key (a key release.) A key identifier that is unique for each key in a keyboard may be recorded for each key action/event so that a sequence of keys pressed may be determined from the data stream from the input device. The typing cadence and typing error component 114 can store the clock time data and key identifier for each key action/event on the user's hard drive, but to greatly enhance security, the preferred embodiment is to store the data temporarily in a RAM of the computing device. The typing cadence and typing error component 114 may intermittently process the data, calculate all the differential timings and determine all the typing errors used later in the process and packages a file it sends to the backend component 106. When the differential timings and typing errors are calculated, as a security measure in some embodiments, the original clock stamps are removed—thus, the order of the characters is removed, making it impossible to put the data back to the original text.

An example of the key action data stream sent to the backend component 106 is contained in Appendix A that is incorporated herein by reference. Appendix A contains an example of a portion of the key action data stream for a particular user (serial number 1234.) As shown in the Appendix, the data may include first key identification data and second key identification data. Thus, for example “8” represents a particular key being pressed or released while “76” represents another key being pressed or released by the user. In one embodiment, the value for each key that identifies the key may be the well-known ASCII value for the particular key. The data also has one or more time samples (TS1, TS2, . . . , TS10, etc) that each happen during a time interval when and after the key combination action occurs. In one embodiment, each time sample may be measured in milliseconds. Each row in the data (other than the header row) represents a particular combination of first and second key actions and then time samples relevant to that particular combination of first and second key actions. When a particular row does not include values for each sample period, the particular combination of first and second key actions has ended and no further key action data about that particular combination of first and second keys is available.

In the key action data in Appendix A, when the first and second key identifier are the same (such as in the first row), then the key action data represents a dwell time for the particular key (such as the key represented by the value “8”) which is a time between a key press of the key and a release of the key by the user. As shown in the portion of the data, there are many different time samples for the dwell time for the key. In the key action data in Appendix A, when the first and second key identifier are different (such as “20” and “8” in the second row), then the key action data represents a flight time between a key press of the first key and a key press of the second key. As shown in the portion of the data, there are often fewer time samples for the flight time between the keys. The system may also detect a key event that is a key being pushed down or released by the user. Thus, a dwell time may be determined. The dwell time is a time period between when a given key is being pushed down (pressed by the user) and that same key is released by the user. Thus, the system and method may detect and use both flight time and dwell time. Thus, the typical cadence data from each computing device (and hence each user who uses that computing device) is captured and processed and used to, among other things, detect a possible neurological disorder of each user of each computing device. For example, the system may gather typing cadence data from users who do not have a neurological disorder and thus can compare the typing cadence data for a person without any neurological disorder to other users.

To generate typing error data, the typing cadence and typing error component 114 may review the sequence of key identifiers included in the key action data to locate patterns of key presses that indicate typing errors. For example, the typing cadence and typing error component 114 may determine a typing error occurred each time the key identifier for the backspace key is detected in the key action data. Other patterns of key presses may also be used to detect typing errors, for example, the typing cadence and typing error component 114 may determine a typing error occurred each time the pattern: typing key identifier, backspace key identifier, typing key identifier is pressed. Additional patterns of key presses may also be used to detect typing errors depending on the configuration of the keyboard used to generate the key action data. The typing key identifier may correspond to any key other than the backspace key, for example, the typing key identifiers may correspond to any alpha numeric key, punctuation key, symbol key, or formatting key (e.g., spacebar or enter) included in the keyboard. Once the typing errors are identified, the typing cadence and typing error component 114 may determine the flight time and dwell time for each key press included in the sequence of keys used to identify the typing error based on the clock time data. For example, the dwell times for the backspace key presses and or typing key presses immediately preceding or following each backspace key press may be determined. The flight time between an error key (i.e., a typing key pressed immediately before the press of a backspace key) and the backspace key and the flight time between the backspace key and a corrective key (i.e., a typing key pressed immediately after the press of a backspace key) to may also be determined.

The typing cadence and typing error component 114 may determine a variety of error measures based on the typing errors that may be used to detect and monitor brain diseases. For example, the typing cadence and typing error component 114 may determine an overall number of errors, an error dispersion, and one or more error consistency measures. The overall number of errors may be determined by comparing the identified errors to all typing included in the key action data. For example, the overall number of errors may be quantified as a number of errors per 1000 keystrokes or other ratio of errors to all typing. To determine an error dispersion typing errors identified by the typing cadence and typing error component 114 may be tagged with one or more labels that describe the typing error. For example, errors may be tagged with an error type based on the key identifiers for the typing keys pressed immediately before and or after the backspace key. In some embodiments, the error types may be very specific with each key sequence that includes a press of a key corresponding to a distinct key identifier followed by a press of the backspace key labeled with a different error type. For example, key sequences that include a press of the key corresponding to the letter “a” followed by a press of the backspace key may be considered a different error type than key sequences that include a press of the key corresponding to the number “3” followed by a press of the backspace key. Error types may also be less specific with key sequences including the press of keys included in groups of key identifiers (e.g., key identifiers that correspond to letters, numbers, punctuation, special characters, enter, spacebar, and the like) followed by a press of the backspace key considered different error types. To determine the error dispersion, the typing cadence and typing error component 114 may determine all of the different types of errors identified in the key action data and or the number of each type of errors based on the labeled error data.

One or more consistency measures may also be determined based on the error data. For example, the typing cadence and typing error component 114 may determine an error consistency, dwell time consistency, and flight time consistency for the identified typing errors. To determine the error consistency, the typing cadence and typing error component 114 may determine an error catalogue for each individual typing activity and or each typing session including multiple typing activities. The error catalogue may include all of the different error types identified in each session and or activity. The error catalogues for different sessions and or activities may be compared to determine the error consistency measures. For example, an error consistency measure from 0 to 1 may be calculated based on the similarity for the typing error catalogues for two typing sessions. High values for the error consistency measure may correspond to more consistent typing performance. For example, if 99 of the 100 typing errors identified in typing session 1 are identified in typing session 2 the error consistency measure determined for the two typing sessions may be 0.99. A baseline error catalogue may also be assembled that includes the error types identified in all of the typing sessions for a user or the most frequent error types (i.e., error types identified at a rate that exceeds a predetermined frequency threshold, for example, at least once for 5 typing sessions, 3 times in at least one session, and the like). The typing cadence and typing error component 114 may determine the error consistency for a typing session by comparing the error catalogue for the typing session to the baseline error catalogue.

Consistency measures may also be determined based on timing data. For example, the typing cadence and error component 114 may determine a dwell time for an error key press that immediately proceeds a key press of the backspace key, a dwell time for a key press of the backspace key, a dwell time for a corrective key press that immediately follows a key press of the backspace key, and the like. The typing cadence and error component 114 may also determine a flight time between an error key press that immediately proceeds a key press of the backspace key and or a flight time between a key press of the backspace key and a corrective key press that immediately follows a key press of the backspace key. The dwell times and or flight times for the error key presses and or corrective key presses may be compared to a baseline dwell time and a baseline flight time respectively. For example, an average of the dwell times for a group of other error key presses (e.g., error key presses during a session, error key presses during the most recent 10 sessions, error key presses during all sessions performed in the last week, error key presses during all sessions performed in the last month, and the like) may be compared to the dwell time for a particular error key press to determine the dwell time error consistency. Similar comparisons of dwell times for presses of the backspace key and presses of corrective keys may also be performed to determine the dwell time error consistency. An average of the flight times for a press of a key selected from a group of error key presses and a press of the backspace key may also be compared to the flight time for a press of a particular errors key and a press of the backspace key determine the flight time error consistency. Similar comparisons for flight times determined for corrective key presses and backspace key presses may also be performed to determine the flight time error consistency.

The overall number of errors, the error dispersion, and or the consistency measures may also be calculated for one error type and or for each type of error. Once determined the error measures may be added to a typing data file. Thus, the typical cadence data and typing error data from each computing device (and hence each user who uses that computing device) is captured and processed and used to, among other things, detect a possible neurological disorder of each user of each computing device. For example, the system may gather typing cadence data from users who do not have a neurological disorder and compare the typing cadence data for a person without any neurological disorder to other users. Additionally, the system may gather typing error data from users who do not have a neurological disorder and compare the typing error data for a person without any neurological disorder to other users.

The typing data file generated by the typing cadence and typing error component 114 may include the key action data and/or the typing cadence data determined form the key action data. For example, the key action data included in the typing data file may have a plurality of key combinations and one or more pieces of timing data about presses of the plurality of key combinations during a continuous typing sequence. The typing data file may also include the typing error data, labeled typing error data including error type and other labels associated with the typing errors identified in the typing error data, and the error measures determined from the typing data. For example, the typing error data included in the typing data file may identify a number and frequency of typing errors committed during a typing session, a library of all of the different typing errors committed during a typing session, and/or typing error measures based on the timing data for the key presses included in the typing sequences identified as typing errors.

In operation, the backend component 106 may act as an organizer and may unpack the typing data file for each computing device, may determine to which user the particular computing device relates based on the file header of the particular typing data file, may convert the data from the format it was sent in into a common format, may calculate the profile data (e.g., the user specific typing cadence and typing error data) for the particular patient and may place the data and profile in the right folder in the data storage.

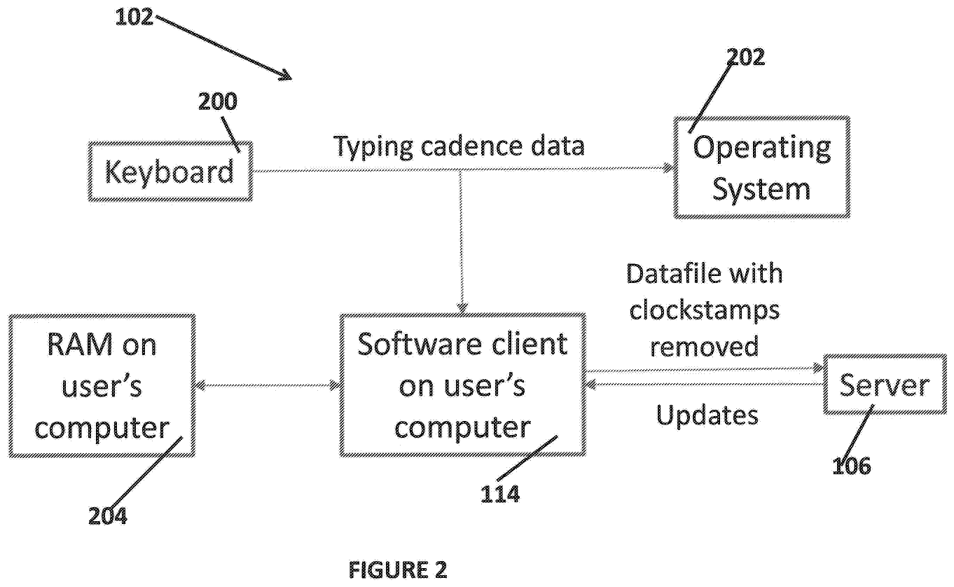

FIG. 2 illustrates an example of an implementation of a computing device 102 that is part of the system for diagnosing and monitoring a neurological disorder. Each computing device 102 may include an input device 200, such as the keyboard as shown in FIG. 2, an operating system 202, memory 204, such as RAM in the computing device and the typing cadence and typing error component 114. The operating system 202 may include, for example, an Apple OS operating system for computers, a UNIX or UNIX like operating system, a Microsoft Windows operating system, an Apple iOS mobile operating system, the Android operating system and/or other tablet and smartphone operating systems. In operation as shown in FIG. 2, the user may use the input device to type and the typing cadence and typing error component 114 (in combination with the operating system 202) may gather data about the typing of the user as well as the typing cadence data and the typing error data of the user which are sent to the typing cadence and typing error component 114. The typing cadence and typing error component 114 may then store the typing cadence data and the typing error data for a period of time and then send a datafile with the clock stamps for the typing cadence data removed (as described above) to the backend component 106. The typing cadence and typing error component 114 may also receive updates from the backend component 106. The updates may include, for example, how many characters the typing cadence and typing error component on each computing device will record prior to sending a typing data file, how many typing errors the typing cadence and typing error component on each computing device will record prior to sending a typing data file, a particular time of day that the client should stop and send a typing data file, etc. Appendix B contains an example of a document that was entered by a user using an input device and Appendix A is an example of a portion of the key action data stream that is generated based on the document in Appendix B being typed by a particular user.

FIG. 3 illustrates an example of an implementation of a backend component 106 that is part of the system for diagnosing and monitoring a neurological disorder. The backend component 106 may have the one or more front end components 108, one or more data stores 110 and one or more data analytics components 112 that are connected to each other as shown in FIG. 2. In this implementation of the backend component and/or the one or more front end components 108 may perform various actions with respect to the incoming key action data and typing error records from each user. For example, the one or more front end components 108 may unpack the key action data stream and typing error records, rename the key action data stream and typing error records, move the key action data stream and typing error records to a designated folder and update a set of log files about the key action data streams and the typing error records. The one or more front end components 108 may be implemented as one or more desktop computers, one or more laptop computers or one or more cloud computing resources and may execute various pieces of software/code including, for example, Windows, Perl, Java Scripts, Microsoft Office and Visual basic. The one or more data stores 110 may be used to store the various key action data streams and typing error records and may segregate the key action data stream and typing error records for each user into a separate storage area, such as a folder for example. The one or more data stores 110 may be implemented in a general purpose computer, specialized computer for high volume storage and complex access or a combination of hardware and software and may execute various pieces of software/code including, for example, Excel and Windows Explorer. The one or more data analytics components 112 may process each typing data file in order to convert the key action data and/or typing error data into a common profile format and then update any statistics and profile about the user as described below in more detail. The one or more data analytics components 112 may be implemented using a general purpose computer and may execute various pieces of software/code including, for example, Excel, other statistical packages, such as SPSS, R, and big data analytics.

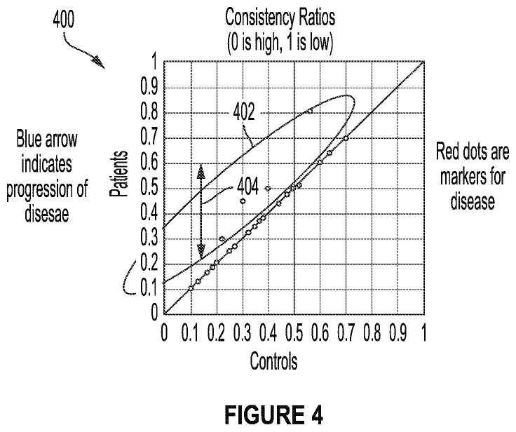

FIG. 4 illustrates an example of the data generated 400 by the system shown in FIG. 1. In particular, the graph 400 shows the consistency ratios for a set of control subjects (that do not have any neurological disorder) and a set of patients with some neurological disorder. The consistency ratios may range from 0 (high consistency ratio) to 1 (low consistency ratio) and may be based on the typing cadence data and the typing error data. For example, the consistency ratios may be determined from the typing error measures described above. As shown in the graph, the control subjects have data values for a key action cadence and/or error consistency measures that are near the midline indicating normal cognitive function. In contrast, the patients have data points 402 above the midline that are markers for a neurological condition. In the graph in FIG. 4, the key action data and/or typing error measures (described elsewhere) may be used to calculate the inconsistency values shown in FIG. 4. For example, a coefficient of variance algorithm may be used as well as other algorithms to determine the inconsistency values. In addition, an arrow 404 superimposed on the graph and its data shows the data values changes that show a progression of the cognitive disease. In various embodiments, the typing error measures generated by the system may be displayed in a chart that displays at least one of the multiple error measures of the user and or at least of one of the consistency measures and or inconsistency values calculated from typing error measures. The user may have a neurological disorder or may not be diagnosed with a neurological disorder. To monitor the disease progression of a patient that has a neurological disorder, at least one of the multiple error measures and/or consistency measures and/or inconsistency values calculated from the typing error measures of a second user that does not have the neurological disorder may be added to the chart. An indication of a progress of the neurological disorder of the user may then be determined based on the distance between the values for the error measures, consistency measures, and/or inconsistency measures of the user having the neurological condition and the values for the error measures, consistency measures, and/or inconsistency measures of the second user that does not have the disease. Larger distances between the values for the user and the second user may indicate more disease progression.

To screen a healthy user for a neurological disorder and/or diagnose a user with a neurological disorder, at least one of the multiple error measures and/or consistency measures and/or inconsistency values calculated from the typing error measures for the healthy user may be displayed in a chart. At least one of the multiple error measures and/or consistency measures and/or inconsistency values calculated from the typing error measures of a second user that has the neurological disorder may be added to the chart. An indication of a diagnosis of the neurological disorder in the healthy user may then be determined based on the distance between the values for the error measures, consistency measures, and/or inconsistency measures of the healthy user and the values for the error measures, consistency measures, and/or inconsistency measures of the second user having the neurological condition. Smaller distances between the values for healthy user and the second user may indicate a positive diagnosis for the neurological disorder and larger distances between the values for the healthy user and the second user may indicate a negative diagnosis for the neurological disorder.

FIG. 5 illustrates an example of the processes 500 performed by each computing device of the system for diagnosing and monitoring a neurological disorder. When the user types a string of characters on the input device (502), the input device records data related to each key event/action that occurs while typing the string. The key events/actions may be either a key press action (key-down) or a release of a key (keyup.) For each such event/action, the input device records the key identifier, the action being performed on the identified key and the clock time in long times, i.e. the time in milliseconds since Jan. 1, 1950. The input device may also record the characters typed for each string. Once the key event/action data and the typed characters have been recorded, the key event data and the typed characters may be sent to the OS (operating system) (504). The typing cadence and typing error component may hook into this communication and extract the data being sent (506) using a standard known API. The data that is extracted by the typing cadence and typing error component may be stored (508) in the computing device. In one embodiment, the data may be stored in the memory of the computing device. When the key action data and/or typed characters stored in the computing device reaches a certain threshold (which can be set by a customer and changed remotely) or other events (such as computer shut down), the typing cadence and typing error component may trigger the sending of a typing data file and the typing cadence and typing error component may prepare the typing data file to be sent to the server (510). During the preparation of the key action data included in the typing data file, the typing cadence and typing error component may calculate differential times, i.e. the time a key is held down and then released (aka dwell time) and the time between key-down on one key and keydown on the next key (aka flight time) based on the extracted key action data. During preparation of the typing error data, the typing cadence and typing error component may determine if the string of recorded typed characters includes a typing error and may determine a number of errors, frequency of errors, or other statistics that describe the typing errors included in a particular string of typed characters. The typing cadence and typing error component may also tag the identified typing errors (e.g., label each error as having a particular, i.e., spelling, grammar, repeated words, repeated characters, and the like, error type). The typing data file including the key action data (and the calculated differential times) and the typing error data then may be sent to the backend component (512.) In some embodiments, typing data file, the key action data, and/or the typing error data from each computing device may be encrypted for security so that the backend component may perform decryption on the typing data files, the key action data, and/or the typing error data. In one embodiment, the key action data including the typing data file may have a format that may include:
(1) Key 1 identification
(2) Key 2 identification (if the same as Key 1, dwell time; if different, flight time; and
(3) Time1, time2, time3, time4, etc. data points for every time a Key1-Key2 sequence occurred.

An example of the key action data having this format is shown in more detail in Appendix A that was discussed above in more detail. Typing error data included in the typing data file may have a format that may include:
(1) Error 1 identification (each error sequence that includes at least one different error key press or corrective key press may have its own error identifier);
(2) Error Sequence (Error Key 1, Backspace Key, Corrective Key 1 . . . ); and
(3) Error time1, error time2, error time3, error time4, etc. data points for the dwell time and flight times for the key presses each time the Error 1 sequence occurred

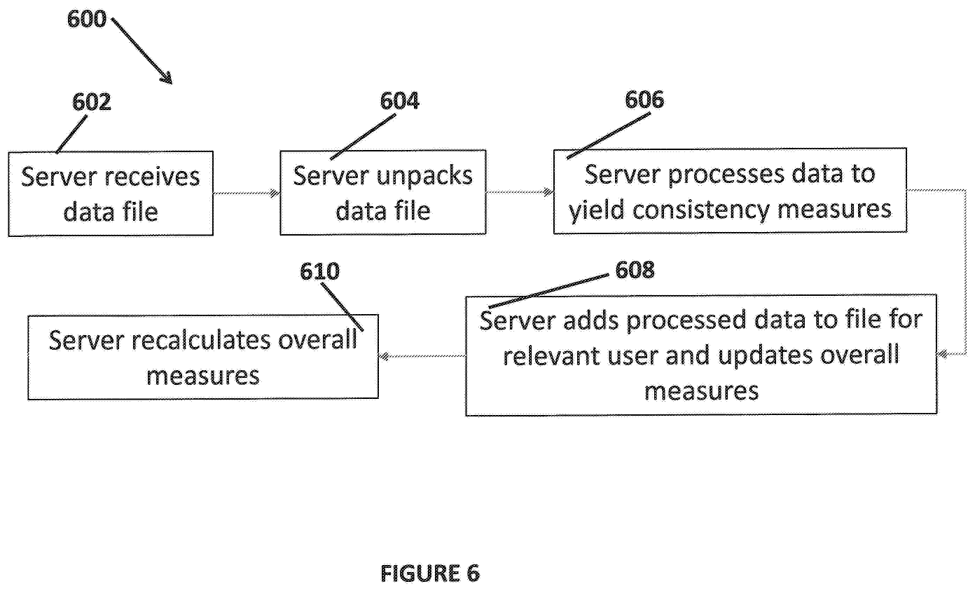

FIG. 6 illustrates an example of the processes 600 performed by the backend component of the system for detecting and monitoring a neurological disorder. The backend component 106 may receive the typing data file (602) and may then unpack the typing cadence data and the typing error data from the typing data files. For example, the typing data files may be in a .bin format and the backend component 106 may convert the typing cadence data and/or typing error data into a .csv format (spreadsheet format) to be read in Excel or other protocols (604). The backend component 106 may process and use the key action cadence data and the typing error data to calculate various statistical properties, including a measure of consistency (606).

The backend component may then add the processed data to a files for the relevant user and update overall measures (608). The overall measures may be, for example, weighted averages determined based on the measure of inconsistency values. The backend component may then recalculate the overall measures (610) which are used to detect and then monitor and/or diagnose a neurological disorder as shown in FIG. 4.

Further details of the above typing cadence system and method are described in U.S. Pat. No. 10,694,987 filed on Jun. 27, 2014 and entitled “Neurological Disorder Determining And Monitoring System And Method”, and U.S. Pat. No. 10,694,947, filed on May 26, 2014 and entitled “System And Method For Continuous Monitoring Of Central Nervous System Diseases, the entirety of which are incorporated by reference herein.

The above system may be part of the system and method for diagnosis and continuous monitoring of CNS diseases. The system for diagnosis and continuous monitoring addresses the need for early and precise diagnosis of CNS diseases. The system uses a method for recording data, processing data locally, sending data to the server and the server's additional processing of data. The system and method addresses the need to provide CNS treating doctors and their patients with accurate, precise and ongoing measurement of cognitive function. Preferably, patients and doctors would be able to see data representing small time increments. The method for recording, preprocessing, sending to server and processing at server is the same as for the above mentioned system implementation described above with reference to FIGS. 1-6. However, the frequency of collecting and processing data, as well as display of data is different.

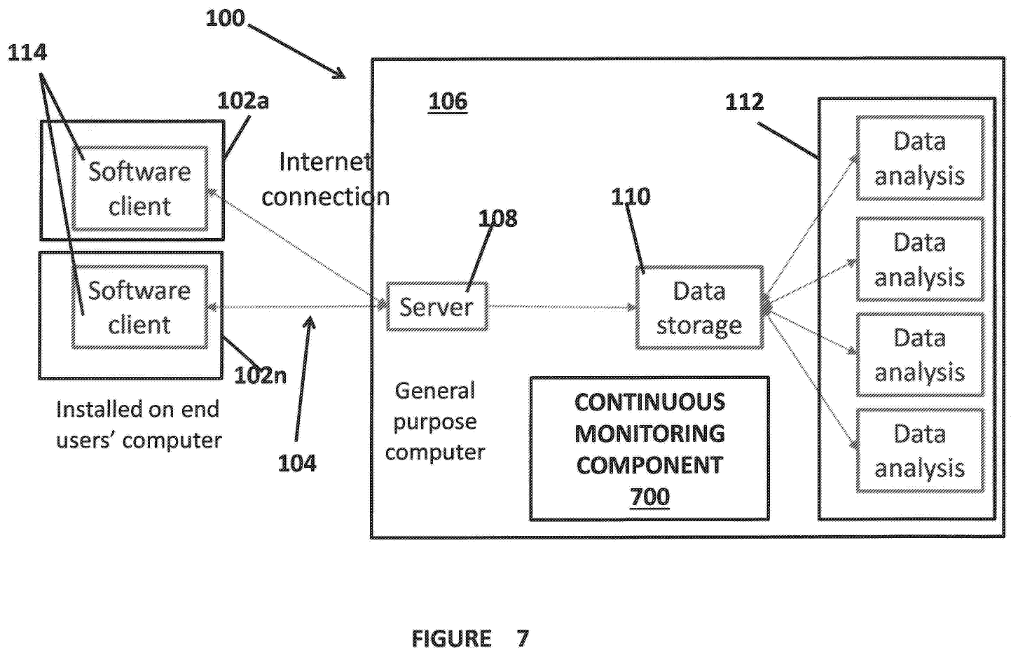

FIG. 7 illustrates a system 100 for diagnosing and monitoring a neurological disorder with a first embodiment of a continuous monitoring component 700 that is integrated into the system 100. In this embodiment, the continuous monitoring component 700 may receive typing cadence data and typing error data that is being sent to the server 108 as described above. The continuous monitoring component 700 may be implemented in hardware or software. When the continuous monitoring component is implemented in hardware, it may be an ASIC, programmable logic device, an integrated circuit, a state machine or a microcontroller that operate to perform the functions and operations of the continuous monitoring component as described below. When the continuous monitoring component is implemented in software, it may be a plurality of lines of code that may be stored on a computer, such as the backend system 106 in FIG. 1 or any other computer system and then executed by a processor of the computer so that the processor is configured to perform the functions and operations of the continuous monitoring component described below.

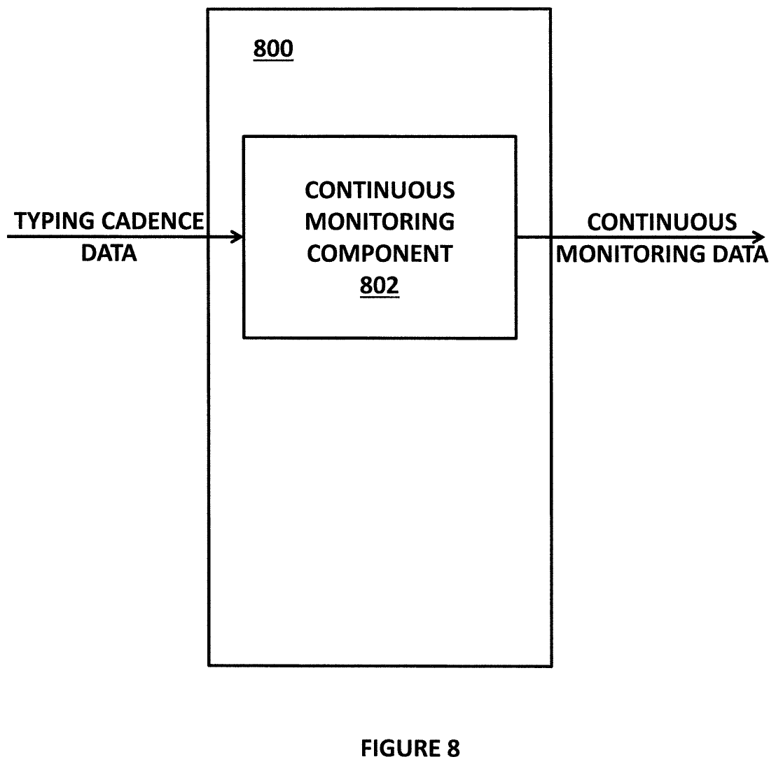

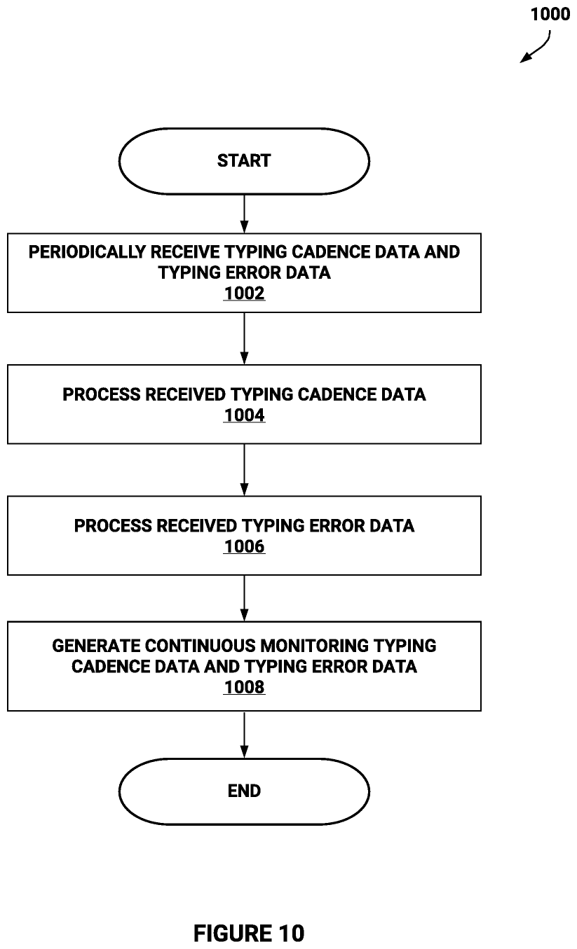

FIG. 8 illustrates a second embodiment of the continuous monitoring system that is a standalone system 800 in which the continuous monitoring component 802 is part of that system 800 that may be a computer system with the usual components. In this embodiment, the continuous monitoring component 802 may receive typing cadence data from an outside source. The continuous monitoring component 802 may be implemented in hardware or software and may perform a series of processes of the algorithm described below in FIG. 10. When the continuous monitoring component 802 is implemented in hardware, it may be an ASIC, programmable logic device, an integrated circuit, a state machine or a microcontroller that operate to perform the functions and operations of the continuous monitoring component as described below. When the continuous monitoring component is implemented in software, it may be a plurality of lines of code that may be stored on a computer, such as the backend system 106 in FIG. 1 or any other computer system and then executed by a processor of the computer so that the processor is configured to perform the functions and operations of the continuous monitoring component described below.

In each of the embodiments shown in FIGS. 7 and 8, typing cadence data and typing error data may arrive at the continuous monitoring component so that the data may be processed and displayed (described in more detail below with reference to FIG. 10). In each of the embodiments shown in FIGS. 7 and 8, the typing cadence data may be periodically received by the continuous monitoring component, such as many times a day or even several times an hour and then displayed as continuous monitoring typing cadence data and continuous monitoring typing error data. The typing cadence data and the typing error data may be gathered, processed and sent to the continuous monitoring component 700, 802 in small increments. The size of the increments is adjustable depending on the application. For example, for building a baseline data set, the system and method may set the increment value at 20000 while, for continuous monitoring, the system and method may set the increment value to 1000 or less.

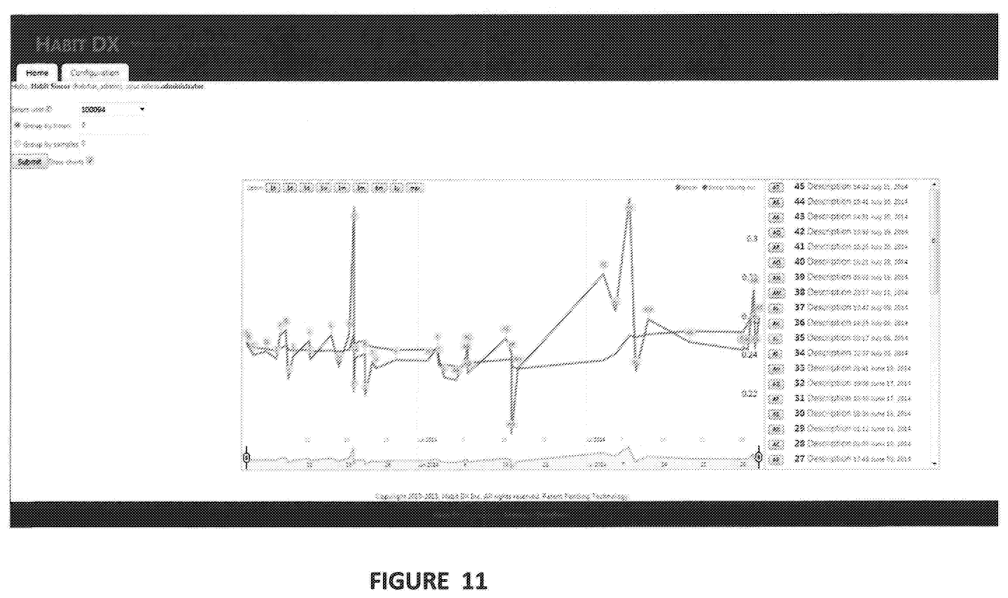

The continuous monitoring component 700, 802 may process the data in the same small increments and add a data point indication, such as a point on a graph or chart for a particular patient whose typing cadence data and typing error data is being received, so that the doctor, patient or patients could view the continuous monitoring output data. FIG. 11 shows an example of a chart generated by the system. In some embodiments, each of the doctor, patient or patients may view the continuous monitoring output data on the computing devices 102 shown in FIG. 7 for example. The embodiment shown in FIG. 8 may also have the capability to allow each doctor, patient or patients to access the system 800 using a computing device 102 (not shown) and then display the continuous monitoring output data.

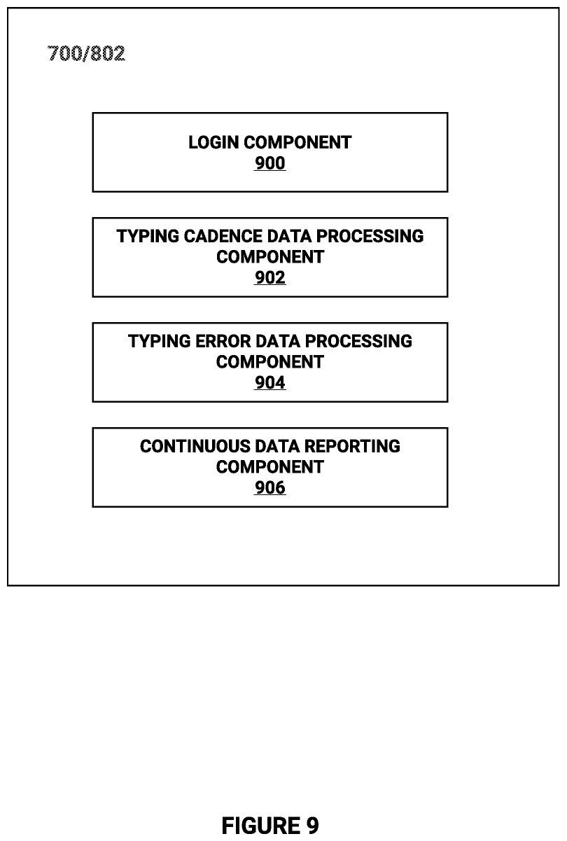

In some embodiments, the system 100, 800 may include a web site (that may be generated and populated in some embodiment, by the continuous data reporting component 904 shown in FIG. 9) that enables users, doctors and patients to view the continuous monitoring output data when the users, doctors and patients are properly authenticated by the system in a known manner. In the case of a doctor accessing the continuous monitoring output data, the doctor may choose which patient's data to view. For a patient with the appropriate authenticated access, the patient can only view their own data. When any user is accessing the continuous monitoring output data, the user may select which time period of data to view and select the form of the display, such as charts, tables or other forms. After all the fields are completed by the user and the request is sent to the system 100, 800, the continuous monitoring component 700, 802 aggregates the relevant data (based on the user request) and displays the data, such as on the web page for the user using, for example, a browser application on each computing device 102.

FIG. 9 illustrates further details of the continuous monitoring component 700, 802 in FIGS. 7 and 8. Specifically, the continuous monitoring component 700, 802 may have a login component 900, a typing cadence data processing component 902, a typing error data processing component 904, and a continuous data reporting component 906. Each of these components may be implemented in software or hardware as described above.

The login component 900 performs a series of processes of a login algorithm that may provide a login user interface with a form that will permit a doctor to register for the continuous monitoring and invite/register all the patients they wish to be measured using the continuous monitoring system. The algorithm of the login component 900 may then communicate information about the doctor and patients to a database/data store 110 of the system. The doctors and patients may use the secure login that the system employs. The login component 900 may implement an authentication method to ensure that both the doctor and the patient are who they are supposed to be.

The typing cadence data processing component 902 may perform the typing cadence data processing as described below with reference to FIG. 10 that uses an algorithm that implements the processes shown in FIG. 10. The typing error data processing component 904 may perform the typing error data processing as described below with reference to FIG. 10 that uses an algorithm that implements the processes shown in FIG. 10. The continuous data reporting component 906 may execute an algorithm to prepare and generate the continuous data reports that may be sent to the authorized users of the system. The continuous data reports may have various forms and may be delivered to the users as a web page on a web site or as a file having a particular data format.

In the continuous data reporting mode, any time another data point was received for the patient (in the case of a patient user) or a patient being treated by the doctor (for a doctor user) at the continuous monitoring component 700, 802, the data reporting, such as a chart, table, web page, etc., would be updated with this most recent data and provided to the user. In another implementation, the data shown to each user by the continuous data reporting component 906 may include not only the raw data, but also a moving average, to highlight for the user where the true extremes are in the captured data. The continuous data reporting component 906 performs a series of processes of a reporting algorithm that may provide typing data and data reports to users of the continuous monitoring system.

FIG. 10 illustrates a continuous monitoring method 1000 that may use the continuous monitoring component to implement the method or the method may be implemented using other elements and components since the method is not limited to operating on/with the continuous monitoring component described above. In the method, typing cadence data and typing error data may be periodically received (1002). The periodicity of the received data may be varied, but may be, for example, several times an hour for a particular patient or a particular doctor that may have one or more patients. The received typing cadence data may be processed (1004). For example, the typing cadence data may be unpacked from the received typing data file for each computing device, the user the particular computing device relates may be determined based on the file header of the particular typing data file, the typing cadence data may be converted from the format it was sent in into a common format, the typing cadence variables included in the profile data for the particular patient may be calculated, and the typing cadence data and profile data may be placed in the right folder in the data storage. The received typing error data may also be processed (1006). For example, the typing error data may be unpacked from the received typing data file for each computing device, the user the particular computing device relates may be determined based on the file header of the particular typing data file, the typing error data may be converted from the format it was sent in into a common format, the typing error measures included in the profile data for the particular patient may be calculated, and the typing error data and profile data may be placed in the right folder in the data storage. The processed typing cadence data may then be used to generate a continuous monitoring typing cadence output data and the processed typing error data may then be used to generate a continuous monitoring typing error output data (1008) that may be, for example, the chart shown in FIG. 4. The continuous monitoring typing cadence output data may also be the raw typing cadence data, a table of the typing cadence data or the exemplary graph shown in FIG. 11. The continuous monitoring typing error output data may also be the raw typed strings that include errors, a typing of the typing error and/or labels associated with each error, or the exemplary graph shown in FIG. 11. The method may repeat the processes 1002-1008 as each new piece/batch of typing cadence data is received.

FIG. 11 shows an example of a chart in a user interface of the system described above that is generated by the system based on the patient data. In the chart, data about the patient is plotted for the patient at different times as shown on the horizontal axis of the chart. The vertical axis of the chart are the values for each time data point of the patient.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings. The embodiments were chosen and described in order to best explain the principles of the disclosure and its practical applications, to thereby enable others skilled in the art to best utilize the disclosure and various embodiments with various modifications as are suited to the particular use contemplated.

The system and method disclosed herein may be implemented via one or more components, systems, servers, appliances, other subcomponents, or distributed between such elements. When implemented as a system, such systems may include an/or involve, inter alia, components such as software modules, general-purpose CPU, RAM, etc. found in general-

purpose computers. In implementations where the innovations reside on a server, such a server may include or involve components such as CPU, RAM, etc., such as those found in general-purpose computers.

Additionally, the system and method herein may be achieved via implementations with disparate or entirely different software, hardware and/or firmware components, beyond that set forth above. With regard to such other components (e.g., software, processing components, etc.) and/or computer-readable media associated with or embodying the present inventions, for example, aspects of the innovations herein may be implemented consistent with numerous general purpose or special purpose computing systems or configurations. Various exemplary computing systems, environments, and/or configurations that may be suitable for use with the innovations herein may include, but are not limited to: software or other components within or embodied on personal computers, servers or server computing devices such as routing/connectivity components, hand-held or laptop devices, multiprocessor systems, microprocessor-based systems, set top boxes, consumer electronic devices, network PCs, other existing computer platforms, distributed computing environments that include one or more of the above systems or devices, etc.

In some instances, aspects of the system and method may be achieved via or performed by logic and/or logic instructions including program modules, executed in association with such components or circuitry, for example. In general, program modules may include routines, programs, objects, components, data structures, etc. that perform particular tasks or implement particular instructions herein. The inventions may also be practiced in the context of distributed software, computer, or circuit settings where circuitry is connected via communication buses, circuitry or links. In distributed settings, control/instructions may occur from both local and remote computer storage media including memory storage devices.

The software, circuitry and components herein may also include and/or utilize one or more type of computer readable media. Computer readable media can be any available media that is resident on, associable with, or can be accessed by such circuits and/or computing components. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic tape, magnetic disk storage or other magnetic storage devices, or any other medium which can be used to store the desired information and can accessed by computing component. Communication media may comprise computer readable instructions, data structures, program modules and/or other components. Further, communication media may include wired media such as a wired network or direct-wired connection, however no media of any such type herein includes transitory media. Combinations of the any of the above are also included within the scope of computer readable media.

In the present description, the terms component, module, device, etc. may refer to any type of logical or functional software elements, circuits, blocks and/or processes that may be implemented in a variety of ways. For example, the functions of various circuits and/or blocks can be combined with one another into any other number of modules. Each module may even be implemented as a software program stored on a tangible memory (e.g., random access memory, read only memory, CD-ROM memory, hard disk drive, etc.) to be read by a central processing unit to implement the functions of the innovations herein. Or, the modules can comprise programming instructions transmitted to a general purpose computer or to processing/graphics hardware via a transmission carrier wave. Also, the modules can be implemented as hardware logic circuitry implementing the functions encompassed by the innovations herein. Finally, the modules can be implemented using special purpose instructions (SIMD instructions), field programmable logic arrays or any mix thereof which provides the desired level performance and cost.

As disclosed herein, features consistent with the disclosure may be implemented via computer-hardware, software and/or firmware. For example, the systems and methods disclosed herein may be embodied in various forms including, for example, a data processor, such as a computer that also includes a database, digital electronic circuitry, firmware, software, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the invention or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the invention, or it may be more convenient to construct a specialized apparatus or system to perform the required methods and techniques.

Aspects of the method and system described herein, such as the logic, may also be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) though again does not include transitory media. Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

Although certain presently preferred implementations of the invention have been specifically described herein, it will be apparent to those skilled in the art to which the invention pertains that variations and modifications of the various implementations shown and described herein may be made without departing from the spirit and scope of the invention. Accordingly, it is intended that the invention be limited only to the extent required by the applicable rules of law.

The system and method described above improve another technology or technical field in that the system and method improves medical diagnosis technology for central nervous system diseases using typing cadence. The system and method improve the technical field of medical diagnosis for central nervous system diseases by providing a continuous monitoring apparatus that receives the typing cadence data and generates a data report based on the received cadence data to provide continuous monitoring of a central nervous system disease of the patient.

The system and method may be implemented using a sensor/input device and a computer system, but the computer system is not performing generic computer functions. Specifically, the computer receives typing cadence data and generates a data report based on the received cadence data to provide continuous monitoring of a central nervous system disease of the patient which are not generic computer functions.

The system and method also cause the transformation of an article to a different state. Specifically, the system receives typing cadence data and provides continuous monitoring based on the typing cadence data. Thus, an article (the typing cadence data) is transformed into continuous monitoring of the central nervous system disease of the patient.

While the foregoing has been with reference to a particular embodiment of the disclosure, it will be appreciated by those skilled in the art that changes in this embodiment may be made without departing from the principles and spirit of the disclosure, the scope of which is defined by the appended claims.

1. A method, comprising:
receiving a typing data file, the typing data file including key action data and a typing error data, the key action data having a plurality of key combinations and one or more pieces of timing data about presses of the plurality of key combinations during a typing sequence, the typing error data identifying at least one error sequence in the key action data;
processing, by a processor of a backend component, the key action data and the typing error data to generate multiple error measures; and
generating diagnostic data, by the processor of the backend component using the generated multiple error measures, about a neurological disorder of a user.

2. The method of claim 1, further comprising determining a typing error catalogue that includes each distinct error sequence identified in the key action data; and
determining an error dispersion that specifies a total number of each distinct error included in the typing error catalogue.

3. The method of claim 1, wherein the at least one error sequence includes at least three key presses and one of the at least three key presses is a press of the backspace key.

4. The method of claim 3, further comprising determining a total number of typing errors in the typing data file based on a number of presses of the backspace key identified in the key action data.

5. The method of claim 1, wherein the key action data includes a plurality of key combinations and one or more pieces of timing data about presses of the plurality of key combinations.

6. The method of claim 5, further comprising determining the multiple error measures based on the one or more pieces of timing data about presses of the plurality of key combinations included in the at least one error sequence.

7. The method of claim 6, wherein the multiple error measures include at least one of a dwell time for a backspace key, a dwell time for an error key, and a dwell time for a corrective key.

8. The method of claim 7, wherein the dwell time is a time period between pressing a first key by a user and a release of the first key by the user.

9. The method of claim 6, wherein the multiple error measures include at least one of a flight time for an error key and a backspace key and a flight time for a backspace key and a corrective key.

10. The method of claim 9, wherein the flight time is a time period between pressing a first key and pressing a second key.

11. The method of claim 1, further comprising generating a chart that displays at least one of the multiple error measures of the user having the neurological disorder, at least one of the multiple error measures of a second user that does not have the neurological disorder, and an indication of a progress of the neurological disorder of the user.

12. The method of claim 1, wherein generating data about the neurological disorder further comprises determining that the neurological disorder exists in the user.

13. The method of claim 12, wherein generating data about the neurological disorder further comprises monitoring the neurological disorder in the user based on the multiple error measures.

14. The method of claim 1, wherein the tying data file is generated from continuous typing of a document.

15. The method of claim 1, wherein the typing data file is generated during a sub-clinical typing activity.

16. A system for diagnosing and monitoring a neurological disorder, the system comprising;
a memory including executable instructions; and
a processor configured to execute instructions and cause the system to:
receive a typing data file, the typing data file including key action data and a typing error data, the key action data having a plurality of key combinations and one or more pieces of timing data about presses of the plurality of key combinations during a typing sequence, the typing error data identifying at least one error sequence in the key action data;
processes the key action data and the typing error data to generate multiple error measures; and
generate diagnostic data using the generated multiple error measures, about a neurological disorder of a user.

17. The system of claim 16, wherein the processor is further configured to receive a continuous stream of typing data and periodically generate a typing data file from the continuous stream of typing data.

18. The system of claim 16, wherein the processor is further configured to determine a typing error catalogue that includes each distinct error sequence identified in the key action data; and
determine an error dispersion that specifies a total number of each distinct error included in the typing error catalogue.

19. The system of claim 16, wherein the processor is further configured to determine the multiple error measures based on the one or more pieces of timing data about presses of the plurality of key combinations included in the at least one error sequence.

20. The system of claim 16, wherein the processor is further configured to generate a chart that displays at least one of the multiple error measures of the user having the neurological disorder, at least one of the multiple error measures of a second user that does not have the neurological disorder, and an indication of a progress of the neurological disorder of the user.
